(19) United States
(12) Patent Application Publication
(10) Pub. No.: US 2012/0290016 A1
(43) Pub. Date: Nov. 15, 2012

(54) Juxta-articular stabilisation system

(76) Inventor: Deepak Kumar, Doncaster (GB)

(21) Appl. No.: 13/574,615

(22) PCT Filed: Jan. 20, 2011
(86) PCT No.: PCT/IB2011/050262

§ 371 Date: Jul. 22, 2012

(30) Foreign Application Priority Data
Jan. 20, 2010 (GB) 1000829.0
Oct. 11, 2010 (GB) 1017027.2

(51) Int. Cl. A61B 17/88; A61B 17/56; A61B 17/80

(52) U.S. Cl. 606/281; 606/289; 606/96

(57) ABSTRACT

The juxta-articular stabilisation system constitutes of a plate with integral pin and tail part, a plate specific jig, a plate and jig specific drill sleeve, a plate positioner, a slotted head screw, a pin bender, screws and pins.

This system can be used for fixation of most types of fractures involving the juxta-articular radius. The plate has a most juxta-articular row of screw holes in individually bendable and detachable extensions especially designed for very distal fractures or the volar lip fractures. The plate and jig assembly have plate positioning apertures in its juxta-articular part to allow adjustment in position of plate in longitudinal, transverse and oblique directions after temporary fixation to the juxta-articular fragment with a pin prior to fixation to diaphyseal fragment. This allows a very precise placement of the plate in the most desirable position. The drill guiding jig can be assembled to the plate prior to surgery thus reducing surgical step and time. The specific orientation of the screws holes in the diaphyseal part of the plate orientates the screws such that when tendons apply forces across the fracture, the plate is wedged between screw and the bone rather than pushed away from the bone. Therefore, more aggressive physical therapy can be commenced earlier and plates with fewer screws in the proximal part can be used without compromising the strength of the fixation. The plate also has bendable and dividable pin part and tail parts on either ends that allows the plate to be used with a chuck or power tool as a pin or drill bit and also have longer purchase into the bone with minimal soft tissue exposure.

International Application Number: PCT/IB2011/050262

International Filing Date: 20 Jan. 2011

International Publication Number: WO 2011/089564 A1

International Publication Date: 28 Jul. 2011

Priority Data:

1000829.0 GB 20 Jan. 2010

1017027.2 GB 11 Oct. 2010

### BACKGROUND

The invention relates to fixation of fractures of distal radius but several of its features may be used in fixation of many fracture especially those close to the joint (juxta-articular). Displaced juxta-articular or intra-articular fractures and many displaced metaphyseal fractures are now commonly treated by operative method (open reduction and internal or external fixation).

Relatively less complex fractures are treated by closed manipulation and stabilisation with K-wires. For more complex fractures especially displaced intra-articular fractures, open reduction and internal fixation with plate and screws has become choice of most surgeons because it allows direct, accurate reduction and early mobilisation of joint. For very few fractures where anatomical reduction is not necessary, intra-medullary implants such as nails are used but they have not gained popularity because such fractures can be successfully treated with less expensive and simpler procedures using k-wires. Moreover, nails are not suitable for the commonly encountered juxta- or intra-articular fractures.

K-wiring, although simpler to use, has several disadvantages. Commonly, it is left exposed out of skin and therefore subjects to the risk of infection. It also requires plaster immobilisation following the procedure as it provides a weak stabilisation on its own.

There are various plating systems available today for fixation of juxta-articular fractures of distal radius. The plates applied on volar aspect with locking screws are very popular.

After a good surgical exposure, precise placement of the plate in the correct position is the most important first step. The quality of bone is often suboptimal especially in the elderly, who sustain the fractures of distal radius most commonly. The best quality of bone is in the subchondral region where screws can have good purchase. This region is very close to the joint, therefore many surgeons trying not to enter into the joint with their screws end up too far proximal in not so good bone and hence, risk failure of fixation. Most plates have an oblong hole in the part that is applied onto the diaphyseal fragment to allow adjustment in plate position. This may offer adjustment after initial fixation to the diaphyseal fragment with one screw through this hole. There are plates that have another transverse oblong hole but again that is in the part that is applied onto the diaphyseal fragment and require fixation to diaphyseal fragment first. Adjustments longer than the length of the oblong hole, a transverse translation of the plate or in longitudinal direction after insertion of screw in the transverse oblong hole are not possible without making another drill hole for another screw. This can certainly cause unnecessary additional weakness in the bone. Furthermore, improvement in fracture reduction becomes extremely difficult if not impossible after fixation to the diaphyseal fragment especially the rotational displacements (supination or pronation of the distal fragment). Therefore, many surgeons choose to do the fixation to the juxta-articular fragment first. This allows them to reduce the displaced juxta-articular fragment better as the attached plate provides better hold on the displaced short fragment. In the technique that involves juxta-articular fixation first, surgeons do not have means of adjustment in plate positioning with most plates currently available. The one plate known to author that allows adjustment over the juxta-articular part is restricted to transverse direction. This requires insertion of a screw in a transversely aligned oblong hole. If the position in longitudinal direction is found to be less than satisfactory after insertion of screw in the said transverse oblong hole, the procedure has to be started all over again.

It is not rare to find fragments adjacent to the main juxta-articular fragments which either have been stabilised only weakly by the screws through the plate or have not been stabilised at all especially when there is a split in the radial styloid process in coronal plane. This fragment is not easily detectable on initial plain roentgenogaphs.

### SUMMARY OF INVENTION

The invention constitutes of a system (a set of devices) for facilitating the use of a bone fracture fixation plate, comprising

means to facilitate adjustment in positioning of the plate on the juxta-articular fragment prior to fixation on the diaphyseal fragment and

means to facilitate longer cortical purchase on bone through smaller exposure, and/or stabilisation of a bone fragment that could not be stabilised by screws in the plate.

The system constitutes of a plate with its plate specific companion drill guiding jig, a jig and plate specific drill sleeve, an eccentric hole plate positioner, a pin bender and a slotted head screw for which we claim novelty. The system also constitutes of screws of various types including top loading screws and pins for which we do not claim novelty.

a) The plate has a juxta-articular part with more than one row of threaded holes, a neck, a shaft or diaphyseal part, a pin and a tail. The juxta-articular part of the plate is twisted in relation to the diaphyseal part. The plate can be further contoured to the anatomy of the surface of the juxta-articular bone during the procedure. The plate has either a group of linked slots or an arrangement of aperture in the plate comprising of at least three holes surrounding an initial-position hole in the middle of juxta-articular part for temporary fixation of the plate to the juxta-articular fragment with one wire and subsequent adjustment in position of the plate in longitudinal, transverse and oblique directions. Once the plate is positioned precisely to the correct place it can be further secured by inserting wires in the wire holes in the juxta-articular part of the plate before insertion of the definitive screws. There are threaded holes in the extensions to the juxta-articular end of the plate and these extensions can be individually bent and readily detached from the plate if required. The neck part has a threaded hole with a recessed collar for the drill guiding jig. The diaphyseal part of the plate has threaded oblique holes directed from juxta-articular side and outer or superficial surface (surface away from bone) to diaphyseal side and inner or deep surface (close to bone) for screws. The pin part is integral to and extends from the diaphyseal end and has sharpening at the end remote from the plate to facilitate insertion into the cortex of the bone. The tail part is integral to and extends from the juxta-articular end of the plate. This can be held in a chuck or a power tool and the pin part penetrates the cortex of the bone like a drill bit. The pin part is bent where it comes out of the bone after said insertion of the pin into the cortex. The said tail is suitable to be inserted in a hole in the region of the outer end of a bone-fixing slotted head screw and is suitable for the tail beyond the screw to be cut off before the tail is fixed in place in such slot. The pin part and tail part can be cut off and the rest of the plate can be used for fracture fixation.

b) The drill guiding jig is plate specific and is configured to sit on a juxta-articular part of the plate. It is attached to the plate with a screw through a hole, which has a proud collar on the undersurface to fit into the recessed collar on the plate. The drill guiding jig has either the group of linked slots or the arrangement of aperture with at least three holes surrounding an initial-position hole corresponding to those into the plate. The jig also has other holes corresponding to the holes in the plate except for those in the bendable and detachable extensions to the juxta-articular end of the plate.

c) The drill sleeve that is specific to the above two, line the apertures in the jig to protect the jig from the drill bit that is guided by these apertures, can lock into the threaded holes of the plate, has scales over its body to indicate how far into the bone a drill bit passing through the sleeve has penetrated and this can be seen from four sides and has means to lock the plate and the jig to a positioning and/or fixing member in an aperture in the plate and the jig during adjustment in position of the plate.

d) Eccentric hole plate positioner allows further fine adjustment in plate position by rotating over a positioning and/or fixing member in an aperture in the plate. The eccentric hole of the plate positioner allows to move the plate in a controlled manner in the desired direction when rotated and has means to temporarily fix the plate to the bone.

e) Two piece pin bender allows the diaphyseal part to be accommodated into the bender while it bends the pin part to allow rest of the plate to sit well onto the bone.

f) Slotted head screw has two holes in its head part, one smooth for the extension from the juxta-articular end of the plate or the tail and the other threaded for the top loading screw. It allows to stabilise a fragment that could not be stabilised by screws in the plate.

### ADVANTAGES

The plate of this system has following advantages over other plates currently available.

1. The group of linked slots or the group of holes in the juxta-articular part allows adjustment in the positioning of the plate transfixed to the juxta-articular fragment of the fractured bone with a pin that remains in its initial position relative to the bone during adjustments in longitudinal, transverse or oblique directions, prior to fixation to the diaphyseal fragment. This facilitates precise placement of the plate in the desired position. There is an opportunity to improve the position even further with the help of eccentric hole plate positioner.

2. The most juxta-articular row of screw holes can be individually bent and detached without having any effect on the rest of the plate. This feature allows fixation of fractures very close to the joint such as volar lip fractures.

3. The pin part, an extension from the diaphyseal end can penetrate bone with minimal soft tissue exposure and allows for longer hold into the bone.

4. Use of a slotted head screw allows stabilisation of a fragment that could not be stabilised by the screws through the plate.

5. The pin part and the tail part can be cut off and the rest can be used as a standard plate.

6. The obliquely placed screw holes in the diaphyseal part of the plate allow screws to be inserted only in a predetermined direction from juxta-articular side and outer surface (surface away from bone) to diaphyseal side and inner surface (close to bone). With the screws in this configuration, the plate is wedged between screws and bone rather than pushed away from the bone by the forces of the tendons across the fracture.

7. The group of linked slots in the drill guiding jig corresponding to those in the plate allows for the jig to be pre-mounted, thus reducing an intra-operative step.

8. The drill sleeve allows for measurement of depth of drill bit into the bone that can be seen from all directions.

### INTRODUCTION TO DRAWINGS

The invention now will be described by referring to the accompanying Figures.

FIG. 1 shows the plate in front profile with adjustment slots

FIG. 2. shows the plate in front profile with adjustment holes

FIG. 3 shows the plate in side profile

FIG. 4 shows the plate in an end profile from the juxta-articular end

FIG. 5 shows the drill guiding jig in front profile

FIG. 6 shows the drill guiding jig in side profile

FIG. 7 shows the drill sleeve in front, side and top end profiles

FIG. 8 shows the slotted head top loading screw in front, side and top end profiles

FIG. 9 shows the eccentric hole plate positioner in front, top and bottom end profiles

FIG. 10 shows both members of the two piece pin bender. A, is front profile of inner member, B is front profile of outer member, C is top end profile when both members are together in an open position and D is top end profile when both members are together in a closed position

FIG. 11 shows the front view of juxta-articular radius fracture stabilised with a plate

FIG. 12 shows the side view of juxta-articular radius fracture stabilised with a plate

### DETAILED DESCRIPTION

Certain juxta-articular bone fracture fixation plates are illustrated and described in

DE 10 2005 043281 A1 (Dieter Marquardt [DE], 15 Mar. 2007),

US 2009/157086 A1 (Digeser Denis [DE] et al, 18 Jun. 2009),

US 2006/173458 A1 (Forstein Micah [US] et al, 3 Aug. 2006)

US 2005/085818 A1 (Huebner Randall J [US], 21 Apr. 2005) and

WO 2007/109437 A2 (Bourda, Marcus [US], 27 Sep. 2007) the entire disclosures of which are hereby explicitly incorporated by reference herein.

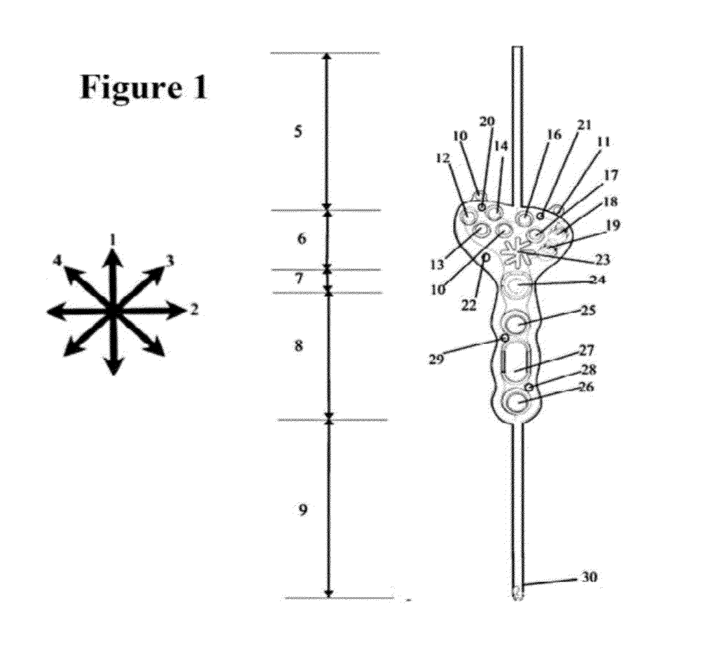

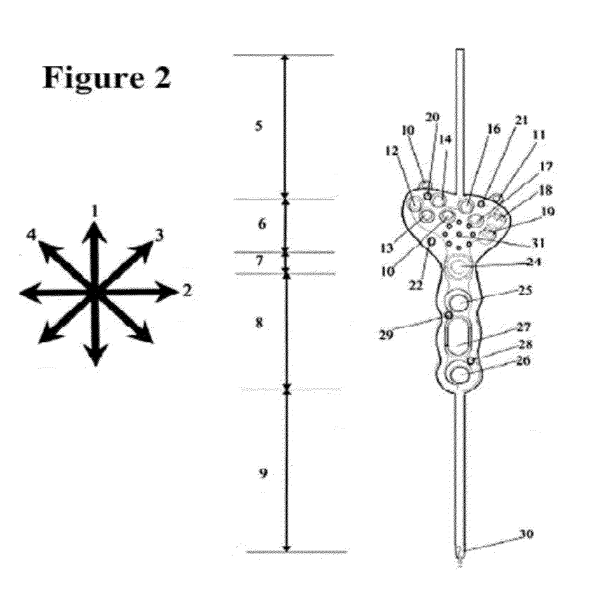

Means 5-9, FIGS. 1, 2 & 3 for facilitating the use of a bone fracture fixation plate 5-9, comprising means 23, 31, FIGS. 1 & 2, to facilitate adjustment in positioning of the plate 5-9 and/or means 30, of part 9 of the plate 5-9, FIGS. 1, 2 & 3 to facilitate insertion into the cortex of the bone 67 and/or part 5 of the plate 5-9, FIGS. 1, 2 & 3 to facilitate holding the plate in a power tool and/or insertion into the hole 57,58, of the slotted head screw 54-61 to facilitate stabilisation of a fragment that could not be stabilised by screws in the plate 5-9 are described below with reference to the above mentioned drawings.

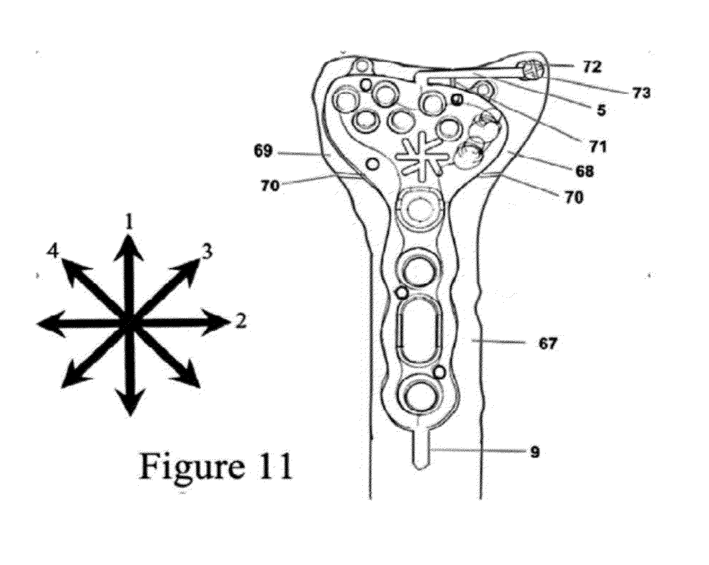

The means 5-9, comprising a bone fracture fixation plate positioning means 23, 31 for facilitating useful, controlled and stable adjustment of the position of the plate 5-9 on a long bone 67, FIGS. 11 & 12, on the juxta-articular side 68, 69 of a fracture 70, during fixation on that side and before fixation or positioning to the bone on the diaphyseal side 67, such positioning means 23, 31 being adapted to facilitate controlled adjustment of position of the plate 5-9 on the bone in longitudinal directions (parallel to the long axis of the limb 1, FIG. 1, 2, 5, 6, 11, 12), transverse directions (that is perpendicular to the long axis, 2, FIG. 1, 2, 4, 5, 11) and oblique directions (that is any direction non-parallel to the long axis, 3,4, FIG. 1, 2, 5, 11);

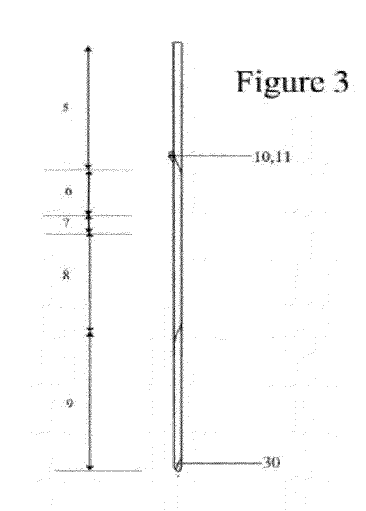

Means 5-9 which comprise the plate 5-9 is shown in FIGS. 1 to 4.

The means 6 has a slot means 23 comprising a cruciform slot arrangement 23 as shown in FIG. 1.

Alternatively, the means 6 has an aperture means 31 comprising an arrangement of holes 31 that comprises of at least three holes surrounding an initial-position hole (the central hole, not numbered) is shown in FIG. 2.

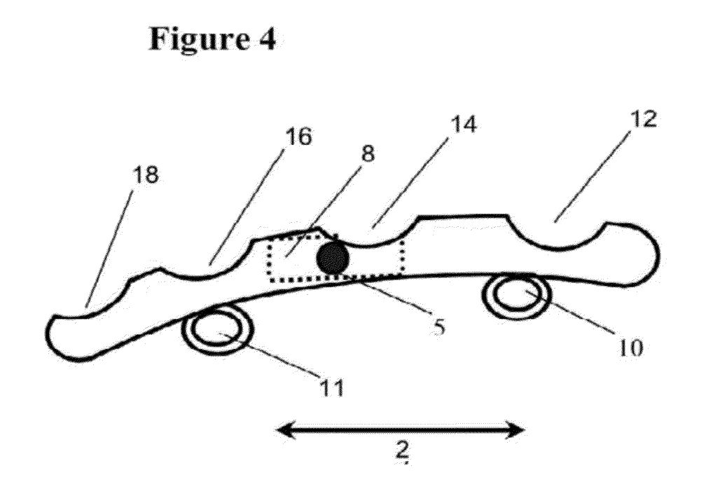

The means 5-9 comprising a bone fracture fixation plate 5-9 has a juxta-articular part 6 and a elongate diaphyseal part 8 connected together and twisted relative to one another is shown in FIG. 4.

The means 6 has extensions 10, 11, FIGS. 1, 2 & 3 on its juxta-articular end and these extensions 10, 11 comprise means adapted to guide and/or hold positioning and/or fixing member/s 80 inserted through the extensions 10, 11 into parts of the bone 68, 69 as shown in FIGS. 11 & 12.

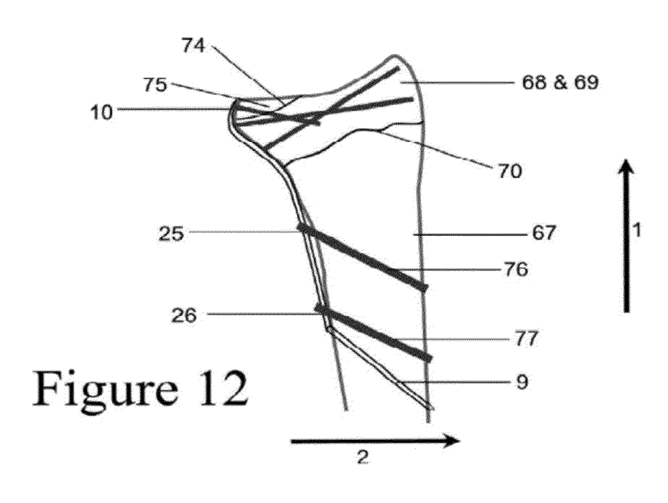

Means 25, 26 FIGS. 1 and 2, comprise of guide means 25, 26 that are directed obliquely towards the bone 67 in a direction away from the fracture 70, for example from juxta-articular side and outer surface (away from bone) to diaphyseal side and inner (close to bone)' and fixing members 76, 77, when inserted into the bone 67 through these guide means 25, 26 follow the above said direction as shown in FIG. 12.

The jig means 43 has a slot means 40 comprising of a cruciform slot arrangement 40 corresponding to the slot arrangement 23 on means 6 of the plate 5-9.

Alternatively, the jig means 43 can have an aperture means comprising an arrangement of holes (not shown) similar to 31 that comprises of at least three holes surrounding an initial-position hole (the central hole, not shown, not numbered).

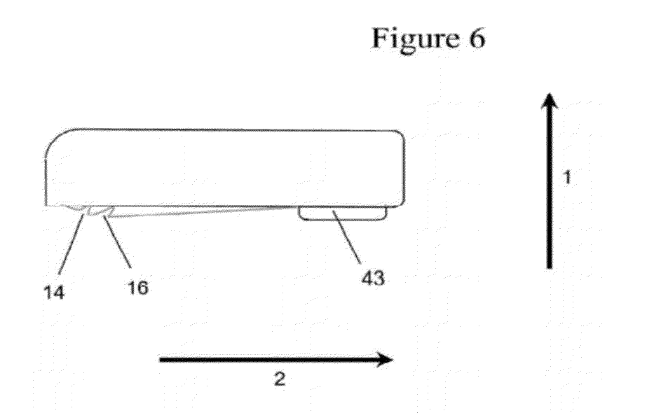

The jig means 43 has a prominent means 44 on its undersurface as shown in FIG. 6, configured to sit into a recession around means 24 of the juxta-articular part 6 of the plate 5-9.

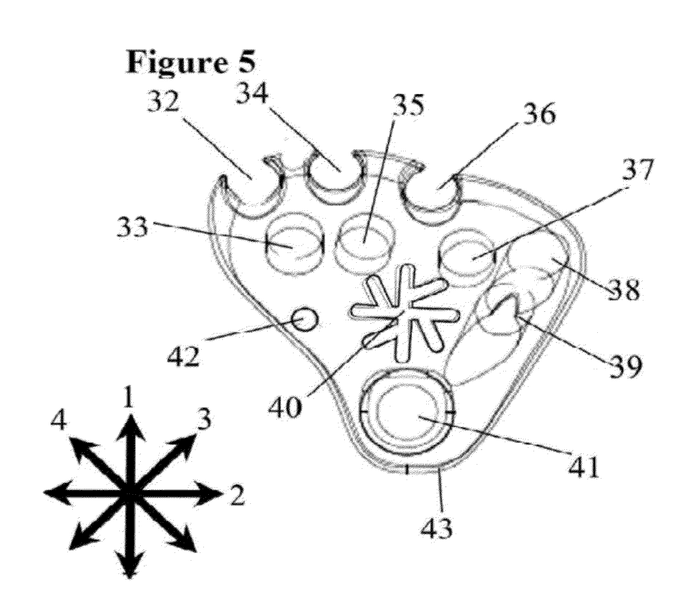

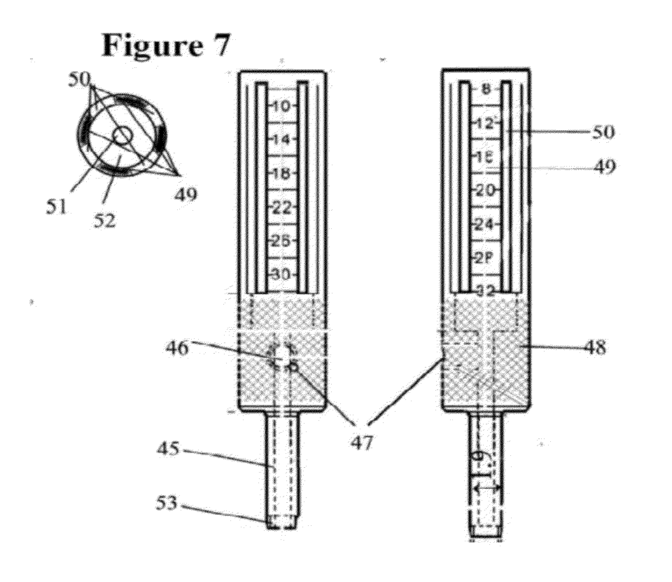

The nozzle part 45 of the sleeve means 48 that lines aperture means 32-39, FIG. 5 in the jig means 43 to protect the jig means 43 from drill bits that they guide is shown in FIG. 7.

The sleeve means 48, comprises means 46 (a side screw hole) and 47 (threads for a side screw), FIG. 7 to lock to the jig means 43 and plate 5-9 to a positioning member in aperture means 40 and 23 or 31.

The sleeve means 48 comprises 4 scale means 49 adapted to indicate through the 4 windows 50 how far into the bone 68, 69 a drill bit passing through the hole 51 in the sleeve means 48 has penetrated is shown in FIG. 7.

Means 48 in which the scale means 49 are adapted to be viewed from all sides is shown in the ‘top plan’ part of FIG. 7.

The drill guiding jig 43 is adapted to be fixed in relation to the plate 5-9 on its neck part 7, FIGS. 1 and 2. This is done by a non-integral fixing member passing through an aperture means 41, FIG. 5 in the jig 43 and engaging in a corresponding aperture 24 in the neck part 7 of the plate 5-9 FIGS. 1 and 2.

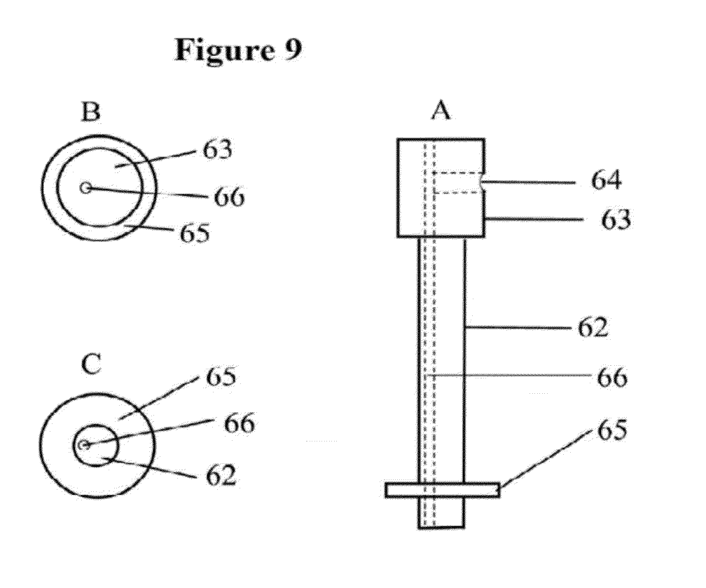

The means 5-9 with the help of a eccentric plate positioner means 62-66, FIG. 9 allows further controlled fine adjustment in the positioning of the plate 5-9 in all directions in the same plane as 1,2,3 & 4 but not limited to 1, 2, 3 & 4.

The means 5-9 comprises of a bone fracture fixation plate 5-9 comprising a plate 6-8 and a pin 9 integral with the plate 5-9 and extending from the end adjacent to diaphyseal part 8 of the plate 6-8, the plate 5-9 being provided with means 30 to facilitate insertion of the pin 9 into the cortex of the bone 67 is shown in FIGS. 1, 2, 3, 11 and 12.

The means 30 comprises of a sharpening at the end of the pin 9 remote from the plate 6-8 as shown in FIGS. 1, 2 and 3. The sharpening is combined with flute (shown but not numbered) and/or threads (not shown) for ease of penetration into the cortex of bone 67 and/or better hold of pin 9 into the bone 67.

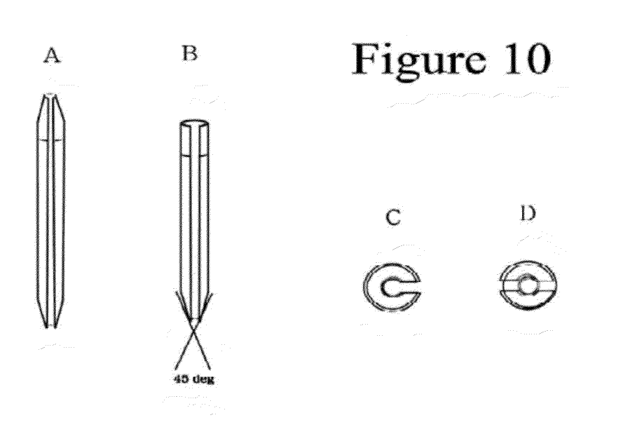

The means 9 of the plate 5-9 is adapted to be bent where it comes out of the bone 67 after said insertion of the pin 9 into the cortex of the bone 67 with the help of a two-piece pin bender A & B shown in FIG. 10.

The plate 5-9 has a tail means 5 that is suitable to be held in a chuck or a power tool to facilitate means 5-9 to be used as a drill bit to facilitate the pin means 9 to penetrate the bone cortex 67, FIGS. 1, 2 & 3.

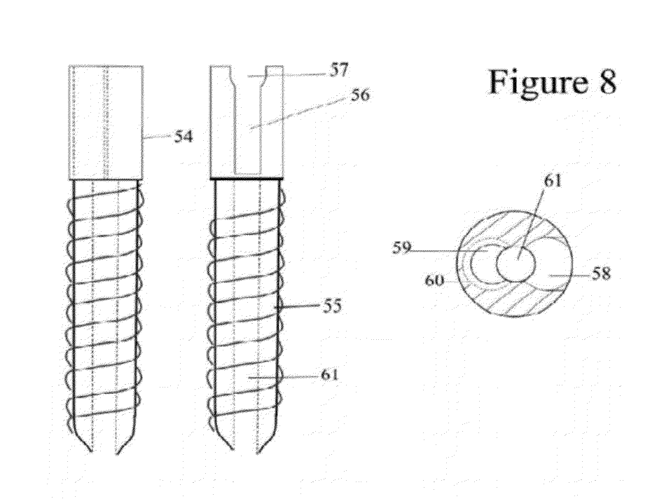

The tail means 5 is suitable to be inserted in a hole means 57, 58, FIG. 8 in the region of the outer end 72, FIG. 11 of a bone-fixing slotted head screw 55 with the help of a top loading screw 73, FIG. 11 and is also suitable to be cut off beyond the outer end 72 of the bone fixing slotted head screw 55 before the tail 5 is fixed in place in such hole 57, 58 as shown in FIG. 11.

The juxta-articular part 6 of the plate 5-9 has apertures 12-19, FIGS. 1 & 2, defined in mutually relatively twisted parts of said plate 5-9 so as to facilitate positioning or fixing members 78, 79, non-integral with the plate, being inserted through such apertures 12-19 and into the bone 68, 69 in mutually non-parallel directions as shown in FIG. 12.

1. A bone fracture fixation system, for facilitating useful, controlled and stable adjustments in positioning a bone fracture fixation plate (6-8) or plate-jig assembly transfixed to the juxta-articular fragment (68,69) of the fractured bone with a pin that remains in its initial position relative to the bone during adjustments prior to fixation to the diaphyseal fragment (67), comprising:
a bone fracture fixation plate (6-8) that has a juxta-articular part (6,7) with or without bendable, dividable extensions (5,10,11) and a diaphyseal part (8) with or without a bendable, dividable extension (9) and an inner surface close to the bone and outer surface away from the bone, a drill guiding jig (43) assembled to the outer surface of the juxta-articular part (6,7) of the plate, both having plurality of apertures of at least two dimensions, one with larger dimension (12-19, 24-27, 32-39, 41) with or without threads for fasteners and the other with smaller dimension (20-22,28,29,42) for pins, located anywhere but usually near the margins for temporarily securing the plate to the bone, characterised in that this plate has additional aperture/apertures (23,31) towards the centre of the juxta-articular part (6,7) away from the margins configured to allow adjustment in longitudinal directions (that is parallel to the long axis of the limb, 1) and/or transverse directions (that is perpendicular to the long axis, 2) and/or oblique directions (that is any direction between longitudinal and transverse axes, 3,4);
a drill sleeve (45-53), a plate positioner (62-66) both adapted to securely hold the position of the plate or plate-jig assembly relative to the bone between adjustments by means of fastener in their side hole (46,64); and
a pin (not illustrated).

2. A plate and a jig as claimed in claim 1, in which the position adjusting aperture is long and narrow like a slot.

3. A plate and a jig as claimed in claim 2, in which the slot is tri-radiate.

4. A plate and a jig as claimed in claim 2, in which the slot is cruciform.

5. A plate and a jig as claimed in claim 4, in which the slot has additional branches to allow position of the plate to be adjusted in directions intermediate to the four branches.

6. A plate and a jig as claimed in claim 4, in which the slot has additional branches emanating from the four branches of the cruciform.

7. A plate and a jig as claimed in claim 1, in which the position adjusting apertures comprise of an arrangement of 4 holes, 3 holes around a central hole.

8. A plate and a jig as claimed in claim 1, in which the position adjusting apertures comprise of an arrangement of 5 holes, 4 holes around a central hole.

9. A plate and a jig as claimed in claim 8, in which there are additional holes to allow position of the plate to be adjusted in directions intermediate to the said four holes.

10. A plate as claimed in claim 1, in which aperture/apertures (25,26) in the diaphyseal part (8) is/are directed obliquely characterised in that this obliquity is from outer surface to inner surface and from juxta-articular end to diaphyseal end, to guide the fasteners (76,77) in the same direction so that they remain parallel and when forces of the tendons across the fracture pull the juxta-articular fragment (68,69) and the plate (6-8) towards the diaphyseal fragment (67), the plate is wedged between these fasteners and the diaphyseal fragment and more firmly applied to the surface of diaphyseal bone.

11. A drill sleeve as claimed in claim 1, in which the laser marking on the drill can be visualised through 4 windows (50) to determine how far drill has traversed into the bone with the help of scale marked on 4 sides (49).

12. A drill sleeve as claimed in claim 11, in which there is a threaded side hole (46) for a fastener so that when the drill sleeve is mounted over a pin transfixing the plate to the bone and the fastener in the side hole is tightened onto the pin while the drill sleeve is pressed down firmly onto the plate, their position relative to each other and bone is held securely.

13. A plate positioner (62-66) as claimed in claim 1, in which there is an eccentric cannulation (66), a flange (65) close to the bottom end and a threaded side hole (64) for a fastener near the top end so that when the positioner is mounted over a pin transfixing the plate to the bone through one of the larger dimension holes (not illustrated) and the positioner is rotated inside the hole over the pin, it can facilitate finer adjustments in the positioning of the plate and when the fastener near the top end is tightened onto the pin while the positioner is pressed down firmly onto the plate, their position relative to each other and bone is held securely.

14. A bone fracture fixation system, for facilitating longer cortical purchase on bone through smaller exposure, and/or stabilisation of a bone fragment that could not be stabilised by fasteners in the plate comprising of:
a bone fracture fixation plate (6-8) that has a juxta-articular part (6,7) and a diaphyseal part (8) and an inner surface close to the bone and outer surface away from the bone, a drill guiding jig (43) assembled to outer surface of the juxta-articular part (6,7) of the plate, both having plurality of apertures for fasteners and pins characterised in that the plate has a bendable, dividable extension (9) with sharp tip (30) from its diaphyseal end to facilitate penetration into the cortex of the bone and a second bendable, dividable extension (5) from the juxta-articular end to facilitate holding the plate with a chuck or a power tool to spin the plate around its long axis like a drill bit;
a slotted head screw (54-61) that is used in combination with the above said second extension (5) from the plate and a top loading screw (73) to stabilise a bone fragment that could not be stabilised by the fasteners in the juxta-articular part (6,7) of the plate, by inserting the slotted head screw (54-61) into this fragment and bending and inserting the tip end of the second extension (5) into the smooth hole (58) in the head part (54) of the slotted head screw and securing its position by tightening the top loading screw (73) into the threaded hole (59) in the head part of the slotted head screw.

15. A plate as claimed in claim 14, in which the extension from the diaphyseal end has a cutting tip (30).

16. A slotted head screw (54-61) as claimed in claim 14, in which there is a central cannulation (61), a shaft part (55) that is threaded on its exterior and a head part (54) that has smooth exterior characterised in that it has two additional holes from the top, one of them is threaded and contained (59), for the top loading screw and the other is smooth (57,58) but may break out of the side wall by less than a portion of the radius, for the tip end of the second extension (5) of the plate (6-8) that can be bend and fed into this hole from the top (72).

17. A method of fixing bone fracture by use of a system, as claimed in claim 14, to facilitate longer cortical purchase on bone through smaller exposure by holding the extension (5) from the juxta-articular end of the plate into a power tool (not illustrated) and driving the extension (9) from the diaphyseal end with its sharp tip (30) into the cortex of the diaphyseal fragment (67) away from the juxta-articular fragment (68,69) at an angle, the plate is bent at the exit point from the bone with the help of a special plate bender (FIG. 10, A-D) to exaggerate the angle to twice the initial value taking care the inner surface of the plate faces away from the bone before bending, the plate is then rotated 180 degrees on the axis of the part that lies within the bone, bringing the inner surface of the plate well apposed onto the surface of the juxta-articular fragment (68,69), drilling the bone through the holes for the fasteners and finally stabilising the fracture by inserting the fasteners; and/or to facilitate stabilisation a fragment that could not be stabilised by the fasteners in the juxta-articular part (6,7) of the plate by inserting a slotted head screw (54-61) into the fragment and bending and inserting the tip end of the extension (5) from the juxta-articular end of the plate into the smooth hole (57,58) of the slotted head screw and securing its position with a top loading screw (72) tightened into the threaded hole (59) of the slotted head screw.

18. A method of adjusting position of a bone fracture fixation plate (6-8) by use of a plate or plate-jig assembly, as claimed in claim 5 or 6, to facilitate useful, controlled and stable adjustments in positioning a bone fracture fixation plate (6-8) or plate-jig assembly transfixed to the juxta-articular fragment (68,69) of the fractured bone with a pin (not illustrated) that remains in its initial position relative to the bone during adjustments are made by translating the plate or plate-jig assembly along the channels of the slot (23) prior to fixation to the diaphyseal fragment (67).

19. A method of adjusting position of a bone fracture fixation plate (6-8) or plate-jig assembly by use of a system, as claimed in claim 13, to facilitate useful, controlled and stable adjustments in positioning a bone fracture fixation plate (6-8) or plate-jig assembly transfixed to the juxta-articular fragment (68,69) of the fractured bone with a pin (not illustrated) that remains in its initial position relative to the bone during adjustments are made by rotating an eccentrically cannulated plate positioner (62-66) over the transfixation pin into a larger dimension hole (not illustrated) of the plate or plate-jig assembly and holding the adjusted position securely by tightening the fastener in the side hole (64) near the top end onto the pin while the positioner is pressed down firmly onto the plate (6-8).

20. A method of adjusting position of a bone fracture fixation plate (6-8) or plate-jig assembly by use of a plate and a jig, as claimed in claim 9, to facilitate useful, controlled and stable adjustments in positioning a bone fracture fixation plate (6-8) or plate-jig assembly transfixed to the juxta-articular fragment (68,69) of the fractured bone with a pin that remains in its initial position relative to the bone during adjustments are made by lifting the plate or plate-jig assembly out of the transfixing pin and putting them back by feeding the pin into another position adjusting hole (31) prior to fixation to the diaphyseal fragment (67).
